(19) United States
(12) Patent Application Publication
(10) Pub. No.: US 2026/0216162 A1
(43) Pub. Date: Jul. 30, 2026

(54) SELECTIVE FIASMA TREATMENT

(71) Applicant: VALO HEALTH, INC., Lexington, MA (US)

(72) Inventors: Mark David Rekhter, Carmel, IN (US); Ella T. Nkhoma, Lexington, MA (US); Paul Brendel, Sewickley, PA (US)

(21) Appl. No.: 19/151,739

(22) PCT Filed: Feb. 1, 2024
(86) PCT No.: PCT/US24/13928

§ 371 Date: Jul. 29, 2025

Related U.S. Application Data
(60) Provisional application No. 63/482,827, filed on Feb. 2, 2023.

(51) Int. Cl. A61K 31/4525; A61K 31/138; A61P 25/22; A61P 25/24

(52) U.S. Cl. CPC A61K 31/4525; A61K 31/138; A61P 25/22; A61P 25/24

(57) ABSTRACT

This disclosure concerns a method of treating or preventing a disease in a subject having atherosclerosis, wherein the disease is treatable or preventable by a functional inhibitor of acid sphingomyelinase (FIASMA) and alternatively by a non-FIASMA. This disclosure also concerns a method of treating or preventing depression or anxiety in a subject having atherosclerosis, wherein the depression or anxiety is treatable or preventable by a FIASMA and alternatively by a non-FIASMA. This disclosure also concerns a method of treating or preventing myocardial ischemia or heart failure in a subject having atherosclerosis, wherein the myocardial ischemia or heart failure is treatable or preventable by a FIASMA and alternatively by a non-FIASMA.

### FIELD

This disclosure concerns a method of treating or preventing a disease in a subject having atherosclerosis, wherein the disease is treatable or preventable by a functional inhibitor of acid sphingomyelinase (FIASMA) and alternatively by a non-FIASMA. This disclosure also concerns a method of treating or preventing depression or anxiety in a subject having atherosclerosis, wherein the depression or anxiety is treatable or preventable by a FIASMA and alternatively by a non-FIASMA. This disclosure also concerns a method of treating or preventing myocardial ischemia or heart failure in a subject having atherosclerosis, wherein the myocardial ischemia or heart failure is treatable or preventable by a FIASMA and alternatively by a non-FIASMA.

### CROSS-REFERENCE TO RELATED APPLICATIONS

This application claims priority to U.S. Provisional Patent Application No. 63/482,827, filed Feb. 2, 2023, the contents of which are hereby incorporated by reference.

### BACKGROUND

Acid Sphyingomyelinase (ASMase; encoded by the SMPD1 gene) hydrolyzes sphingomyelin, resulting in the generation of ceramide and phosphorylcholine. Ceramides play an important role in signal transduction in programmed cell death (apoptosis), the cell cycle, and cell differentiation and senescence. The presence of excess ceramides is causal in many diseases, including depression and cancer.

A broad group of compounds have shown a high potential to inhibit ASMase without direct inhibition (1). These compounds are called functional inhibitors of acid sphyingomyelinase (FIASMAs) and have been proposed to act via an alternative mechanism. In defining these FIASMAs as having the ability to reduce the activity of ASMase by 50% at a 10 UM concentration, Kornhuber et al. were able to identify 72 different compounds that operate as FIASMAs (2). Examples of FIASMAs include common antidepressants, antihistamines, calcium channel blockers, among several other classes of medications.

### SUMMARY

In a first aspect, there is provided a method of treating or preventing a disease in a subject having atherosclerosis, wherein the disease is treatable or preventable by a functional inhibitor of acid sphingomyelinase (FIASMA) and alternatively by a non-FIASMA. The method comprises selectively administering to the subject a FIASMA for the treatment or prevention of the disease and not administering to the subject a non-FIASMA for the treatment or prevention of the disease.

The inventors have found that patients with some degree of atherosclerosis (e.g., at least 50 years old and/or diagnosed with atherosclerosis/coronary artery disease) are at a lower risk of death and major cardiac adverse events if they receive a FIASMA for the treatment of a disease instead of a non-FIASMA. This finding advantageously means that a FIASMA can be selectively administered for a disease if the patient has atherosclerosis, thus reducing the risk of patient death and major adverse cardiac events.

In a second aspect, there is provided a method of treating or preventing depression or anxiety in a subject having atherosclerosis, wherein the depression or anxiety is treatable or preventable by a functional inhibitor of acid sphingomyelinase (FIASMA) and alternatively by a non-FIASMA. The method comprises selectively administering to the subject a FIASMA for the treatment or prevention of the depression or anxiety and not administering to the subject a non-FIASMA for the treatment or prevention of the depression or anxiety.

The inventors have advantageously demonstrated that treatment of depression/anxiety in a patient with FIASMA instead of non-FIASMA reduces the risk of death of the patient when the patient has atherosclerosis (e.g., at least 50 years old and/or diagnosed with atherosclerosis/coronary artery disease). This finding advantageously means that a FIASMA can be selectively administered for the treatment or prevention of depression or anxiety if the patient has atherosclerosis, thus reducing the risk of patient death.

In a third aspect, there is provided a method of treating or preventing a disease in a subject having atherosclerosis, wherein the disease is treatable or preventable by a functional inhibitor of acid sphingomyelinase (FIASMA) and alternatively by a non-FIASMA, wherein subject is being administered a non-FIASMA for the treatment or prevention of the disease. The method comprises ceasing administration of the non-FIASMA for the treatment or prevention of the disease, and selectively administering to the subject a FIASMA for the treatment or prevention of the disease and not administering to the subject a non-FIASMA for the treatment or prevention of the disease.

In a fourth aspect, there is provided a method of treating or preventing a disease in a subject having atherosclerosis, wherein the disease is treatable or preventable by a functional inhibitor of acid sphingomyelinase (FIASMA) and alternatively by a non-FIASMA. The method comprises determining that the patient is at elevated risk of MACE, and selectively administering to the subject a FIASMA for the treatment or prevention of the disease and to reduce the risk of MACE, and not administering to the subject a non-FIASMA for the treatment or prevention of the disease.

In a fifth aspect, there is provided a method of treating or preventing a disease in a subject having atherosclerosis, wherein the disease is treatable or preventable by a functional inhibitor of acid sphingomyelinase (FIASMA) and alternatively by a non-FIASMA. The method comprises selectively administering to the subject a FIASMA for the treatment or prevention of the disease and to reduce the risk of MACE or avoid an increased risk of MACE, and not administering to the subject a non-FIASMA for the treatment or prevention of the disease.

In a sixth aspect, there is provided a method of reducing the risk of a MACE in a subject, wherein the subject has atherosclerosis and is being treated for a secondary disease that is not atherosclerosis. The method comprises determining that the subject is at elevated risk for MACE, and selectively administering a FIASMA for the treatment or prevention of the secondary disease and to reduce the risk of MACE, and not administering to the subject a non-FIASMA for the treatment or prevention of the secondary disease.

The foregoing and other objects, features and advantages of the invention will become more apparent from the following detailed description, which proceeds with reference to the accompanying figures.

### BRIEF DESCRIPTION OF THE DRAWINGS

FIGS. 1A and 1B show (A) a covariate balance plot and (B) propensity score density for cohort A (patients with presumed atherosclerosis).

FIGS. 2A and 2B show Kaplan-Meier survival plots of the time to all-cause mortality of patients with presumed atherosclerosis (cohort A) and using FIASMA SSRIs versus non-FIASMA SSRIs. (A) Survival plot under survival assumption 1. (B) Survival plot under survival assumption 2.

FIGS. 3A and 3B show Kaplan-Meier survival plots of the time to a major adverse cardiac event (MACE) of patients with presumed atherosclerosis (cohort A) and using FIASMA SSRIs versus non-FIASMA SSRIs. (A) Survival plot under survival assumption 1. (B) Survival plot under survival assumption 2.

FIGS. 4A and 4B show (A) a covariate balance plot and (B) propensity score density for cohort B (patients with confirmed atherosclerosis).

FIGS. 5A and 5B show Kaplan-Meier survival plots of the time to all-cause mortality of patients with confirmed atherosclerosis/CAD (cohort B) and using FIASMA SSRIs versus non-FIASMA SSRIs. (A) Survival plot under survival assumption 1. (B) Survival plot under survival assumption 2.

FIGS. 6A and 6B show Kaplan-Meier survival plots of the time to a major adverse cardiac event (MACE) of patients with confirmed atherosclerosis/CAD (cohort B) and using FIASMA SSRIs versus non-FIASMA SSRIs. (A) Survival plot under survival assumption 1. (B) Survival plot under survival assumption 2.

### DETAILED DESCRIPTION

### I. Abbreviations

ASMase: acid sphyingomyelinase
CACS: coronary artery calcium score
CAD: coronary artery disease
FIASMA: functional inhibitor of acid sphingomyelinase
IMT: intima-media thickness
MACE: major adverse cardiac event
MI: myocardial infarction
SSRI: selective serotonin reuptake inhibitor

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds), The Encyclopaedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administering and selectively administering: As used herein, administering an agent (e.g., a FIASMA, or a non-FIASMA) to a subject means to give, apply or bring the agent into contact with the subject. Administration can be accomplished by any of a number of routes depending on the agent being administered, and include, for example, oral, topical, intranasal, inhalation, intravenous, intramuscular, intravitreal, conjunctival, intracorneal, intraocular, ophthalmic, subcutaneous, vaginal, rectal, and intraperitoneal. “Selectively 10 administering” means choosing to administer a first agent (e.g., a FIASMA) instead of administering a second agent (e.g., a non-FIASMA) for the treatment or prevention of a disease.

Atherosclerosis: A thickening or hardening of the arteries caused by a build-up of plaque in the inner lining of an artery.

Coronary artery disease (CAD): A disease caused by plaque build-up in the walls of the coronary arteries overtime which narrows the arteries and restricts or entirely blocks the blood flow to the heart (i.e., atherosclerosis of the coronary arteries).

Functional inhibitor of acid sphingomyelinase (FIASMA): compounds having the ability to reduce the activity of acid sphyingomyelinase (ASMase) by 50% at a 10 UM concentration. A number of known FIASMAs are detailed in Kornhuber et al. (Functional inhibitors of acid sphingomyelinase (FIASMAs). Handb Exp Pharmacol. 2013; (215): 169-186. doi: 10.1007/978-3-7091-1368-4_9), which is hereby incorporated by reference in its entirety. Determining percentage inhibition of an enzyme is routine in the art. A method of determining inhibition of ASMase is described in Kornhuber et al. (Identification of novel functional inhibitors of acid sphingomyelinase. PLOS One. 2011; 6 (8): e23852. doi: 10.1371/journal.pone.0023852), which is hereby incorporated by reference in its entirety. Briefly, the activity of ASMase was determined in whole cell lysates. After the substance was added to the growth medium at a final concentration of 10 UM, cells were kept at 37° C. in a humidified atmosphere at 8.5% CO₂ for 30 minutes (DMEM: pH 7.5). Residual ASM activity was normalized to control cells treated with the solvent alone. Non-FIASMAs include compounds which do not have the ability to reduce the activity of ASMase by 50% at a 10 UM concentration. In some embodiments, a non-FIASMA is a compound which reduces the activity of ASMase by less than 50% at a 10 UM concentration. In some embodiments, a non-FIASMA is a compound which reduces the activity of ASMase by 1-49% at a 10 UM concentration. In some embodiments, a non-FIASMA is a compound which reduces the activity of ASMase by 10-45% at a 10 UM concentration.

MACE: a major adverse cardiac event which is an important composite clinical measure of efficacy and safety outcomes for a patient. A MACE includes, for example, myocardial infarction, stroke, unstable angina, heart failure, hospitalisation for heart failure, acute coronary syndrome, ischemic heart disease, revascularization procedures, cardiovascular death, and all-cause mortality.

Pharmaceutically acceptable: The term “pharmaceutically acceptable” refers to a non-toxic material that does not interfere with the effectiveness of an active pharmaceutical ingredient(s).

Pharmaceutically acceptable salt: A salt that is prepared from inorganic acids such as hydrochloric acid, hydrobromic acid, hydroiodic acid, phosphoric acid, metaphosphoric acid, nitric acid, or sulfuric acid, and/or from organic acids such as formic acid, acetic acid, trifluoroacetic acid, benzenesulfonic acid, benzoic acid, citric acid, ethanesulfonic acid, fumaric acid, gluconic acid, glycolic acid, lactic acid, maleic acid, malic acid, methanesulfonic acid, succinic acid, p-toluenesulfonic acid, or tartaric acid by methods known in the art.

Subject: The terms “subject” and “patient” are used interchangeably herein, and is meant an animal to be identified or treated in the methods described herein. In many embodiments, the subjects are mammals, particularly primates, with human subjects being preferred.

Therapeutically effective amount: A dose sufficient to provide a therapeutic benefit in the treatment or prevention of a disease, or to delay, minimize, or reduce the rate of advancement of one or more symptoms associated with the disease, or to cause regression of the disease.

Treating or preventing a disease: Treating a disease in a subject means that one or more symptoms of the disease are ameliorated, i.e., are less severe than before the treatment, and/or that progression of the disease is prevented (i.e., the disease does not advance such that the disease is stabilized) or slowed (i.e., the disease does not advance as quickly to the next stage of the disease). It does not necessarily mean that the symptoms of the disease are completely remedied so that they are no longer present in the subject, although in some methods, this may be the case. Preventing a disease in a subject means that the treatment is prophylactic in terms of completely or partially preventing the disease or one or more symptoms thereof.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Methods

### A. Diseases Treatable or Preventable by a FIAMSA and Alternatively by a Non-FIASMA

Described herein is a method of treating or preventing a disease in a subject having atherosclerosis, wherein the disease is treatable or preventable by a functional inhibitor of acid sphingomyelinase (FIASMA) and alternatively by a non-FIASMA, wherein the method comprises selectively administering to the subject a FIASMA for the treatment or prevention of the disease and not administering to the subject a non-FIASMA for the treatment or prevention of the disease.

The disease may be any disease that is treatable or preventable by a FIASMA and alternatively by a non-FIASMA. In other words, there are at least two therapies that can be administered to treat or prevent the disease, including a FIASMA and a non-FIASMA; there is accordingly a choice between administering the FIASMA or administering the non-FIASMA to the subject. The method comprises selectively administering to the subject a FIASMA for the treatment or prevention of the disease and not administering to the subject a non-FIASMA for the treatment or prevention of the disease. In other words, the choice has been made to administer the FIASMA for treating or preventing the disease instead of administering the non-FIASMA for treating or preventing the disease.

Typically, selectively administering the FIASMA to the subject involves administering a therapeutically effective amount of the FIASMA to the subject.

In some embodiments, the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome. In particular embodiments, the disease is depression or anxiety. Further information relating to these embodiments is provided in subsection B below.

In some embodiments, the disease is not atherosclerosis or a disease caused by atherosclerosis. Diseases caused by atherosclerosis include, for example, myocardial infarction, heart failure, stroke, aneurysm, blood clotting, angina, coronary heart disease, peripheral arterial disease, carotid artery disease, and chronic kidney disease.

The FIASMA may be any suitable FIASMA that is used to treat or prevent a particular disease. For example, when the disease is depression, the FIASMA will be a FIASMA that is used to treat or prevent depression. As a further example, when the disease is heart failure, the FIASMA will be a FIASMA that is used to treat or prevent heart failure. Various FIASMAs are known and are reported in Table 1 of Kornhuber et al. (Functional inhibitors of acid sphingomyelinase (FIASMAs). Handb Exp Pharmacol. 2013; (215): 169-186. doi: 10.1007/978-3-7091-1368-4_9), which is hereby incorporated by reference in its entirety.

In some embodiments, the FIASMA is selected from the group consisting of: Alverine, Amiodarone, Amitriptyline, Amlodipine, Aprindine, Astemizole, AY-9944, Camylofine, Carvedilol, Cepharanthine, Benztropine, Bepridil, Biperiden, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

In various embodiments, the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine. In some embodiments, the FIASMA is a tricyclic antidepressant (e.g., Amitriptyline, Clomipramine, Desipramine, Doxepin, Imipramine, Nortriptyline, Protriptyline, Trimipramine). In some embodiments, the FIASMA is a selective serotonin reuptake inhibitor (SSRI). In some embodiments, the FIASMA is Paroxetine or Fluoxetine. In various embodiments, the FIASMA is Fluoxetine.

It is noted that citalopram and escitalopram are incorrectly listed as FIASMAs in certain prior art documents, including in Hoertel et al. (Association Between FIASMAs and Reduced Risk of Intubation or Death in Individuals Hospitalized for Severe COVID-19: An Observational Multicenter Study. 2021. Clin. Pharmacol. Ther., 110:1498-1511). However, these drugs are not considered to be FIASMAs within the scope of the present application because they do not have the ability to reduce the activity of acid sphyingomyelinase (ASMase) by 50% at a 10 μM concentration.

In some embodiments, the non-FIASMA that is not administered is a non-FIASMA SSRI (e.g., citalopram, escitalopram, vilazodone, vortioxetine) or a non-FIASMA Serotonin and norepinephrine reuptake inhibitor (SNRI) (e.g., duloxetine, venlafaxine, desvenlafaxine) or a non-FIASMA monoamine oxidase inhibitor (MAOI).

In some embodiments, the non-FIASMA that is not administered is a non-FIASMA calcium channel antagonist (e.g., nifedipine, diltiazem, verapamil).

In some embodiments, the non-FIASMA that is not administered is a non-FIASMA antihistamine (e.g., diphenhydramine, acrivastine, cetirizine, fexofenadine).

Atherosclerosis is a thickening or hardening of the arteries caused by a build-up of plaque in the inner lining of an artery. The atherosclerosis may affect any artery of the body. For example, the atherosclerosis may affect the arteries of the heart (causing coronary artery disease), the arteries of the legs, arms or pelvis (causing peripheral artery disease), the arteries of the neck (causing carotid artery disease), the arteries supplying blood to the kidneys (causing renal artery stenosis), the arteries supplying blood to the brain (causing vertebral artery disease) and/or the arteries supplying blood to the intestines (causing mesenteric artery ischemia). In various embodiments, the subject has coronary artery disease. Coronary artery disease is caused by plaque build-up in the walls of the coronary arteries overtime which narrows the arteries and restricts or entirely blocks the blood flow to the heart (i.e., atherosclerosis of the coronary arteries).

The subject is considered to have atherosclerosis if the subject is diagnosed with atherosclerosis (i.e., confirmed atherosclerosis) and/or it is inferred that the subject has atherosclerosis if the subject meets a certain criterion or criteria (i.e., presumed atherosclerosis).

The subject may be diagnosed with atherosclerosis using one or more diagnostic methods. Diagnostic methods for atherosclerosis include blood tests (e.g., blood sugar or cholesterol levels), electrocardiograms, ultrasound imaging (e.g., 2D or 3D vascular ultrasound), ankle-brachial index, cardiac angiography, computerised tomography scans, magnetic resonance angiography and positron emission tomography.

It may be inferred that the subject has atherosclerosis if the subject meets a certain criterion or criteria. In various embodiments, the subject has atherosclerosis if the subject is at least 50 years old. In particular embodiments, the subject has atherosclerosis if the subject is at least 55 years old. In some embodiments, the subject has atherosclerosis if the subject is at least 60 years old. In some embodiments, the subject has atherosclerosis if the subject has previously had a myocardial infarction. Typically, the subject has atherosclerosis if the subject is at least 50 years old.

In some embodiments, the subject has family history of myocardial infarction. In some embodiments, the subject has previously had a myocardial infarction. In some embodiments, the subject is at least 50 years old and/or the subject has family history of myocardial infarction and/or the subject has previously had a myocardial infarction.

The subject may be any subject. For example, the subject may be a human or a non-human animal. In some embodiments, the subject is a human. In some embodiments, the subject is a non-human animal. In some embodiments, the subject is a non-human animal selected from the group consisting of primate, horse, sheep, pig, cow, mouse, rat, rabbit, dog, and cat.

In some embodiments, the method comprises not administering to the subject a non-FIASMA for the treatment or prevention of any disease or disorder. In other words, in some embodiments the method does not comprise administering to the subject a non-FIASMA for the treatment or prevention of any disease or disorder. Accordingly, the subject may be defined as a subject who has not been administered a non-FIASMA.

### B. Depression or Anxiety

Described herein is a method of treating or preventing depression or anxiety in a subject having atherosclerosis, wherein the depression or anxiety is treatable or preventable by a FIASMA and alternatively by a non-FIASMA, wherein the method comprises selectively administering to the subject a FIASMA for the treatment or prevention of the depression or anxiety and not administering to the subject a non-FIASMA for the treatment or prevention of the depression or anxiety.

In some embodiments, the method is a method of treating or preventing depression. Depression is a common mental health disorder with multiple subtypes. The depression may be any type/category of depression provided that the depression is treatable or preventable by a FIASMA and alternatively by a non-FIASMA. The depression may be selected from the group consisting of: clinical depression, depressive episode, recurrent depressive disorder, reactive depression, dysthymia, cyclothymia, manic depression, psychotic depression, prenatal depression, postnatal depression and seasonal affective disorder.

In some embodiments, the disease is anxiety. Anxiety is a common mental health disorder with multiple subtypes. The anxiety may be any type/category of anxiety, provided that the anxiety is treatable or preventable by a FIASMA and alternatively by a non-FIASMA. The anxiety may be selected from the group consisting of: generalised anxiety disorder, obsessive-compulsive disorder, panic disorder, post-traumatic stress disorder and social phobia.

The FIASMA may be any suitable FIASMA that is used to treat or prevent depression or anxiety. For example, when the disease is depression, the FIASMA will be a FIASMA that is used to treat or prevent depression. Various FIASMAs are known and are reported in Table 1 of Kornhuber et al. (Functional inhibitors of acid sphingomyelinase (FIASMAs). Handb Exp Pharmacol. 2013; (215): 169-186. doi: 10.1007/978-3-7091-1368-4_9).

In various embodiments, the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine. In some embodiments, the FIASMA is a selective serotonin reuptake inhibitor (SSRI). In some embodiments, the FIASMA is Paroxetine or Fluoxetine. In various embodiments, the FIASMA is Fluoxetine.

In some embodiments, the method comprises not administering to the subject a non-FIASMA for the treatment or prevention of any disease or disorder. In other words, in some embodiments the method does not comprise administering to the subject a non-FIASMA for the treatment or prevention of any disease or disorder.

In particular embodiments, there is provided a method of treating or preventing depression or anxiety in a subject having atherosclerosis, wherein the depression or anxiety is treatable or preventable by a FIASMA SSRI (e.g., Fluoxetine and/or Paroxetine) and alternatively by a non-FIASMA SSRI (e.g., Escitalopram and/or Citalopram), and wherein the method comprises selectively administering to the subject the FIASMA SSRI for the treatment or prevention of the depression or anxiety and not administering to the subject the non-FIASMA SSRI for the treatment or prevention of the depression or anxiety.

The above description in relation to the methods of treating or preventing a disease in a subject is equally applicable to these methods of treating or preventing depression or anxiety in a subject.

### C. Second Medical Uses

Also described herein is a functional inhibitor of acid sphingomyelinase (FIASMA) for use in treating or preventing a disease in a subject having atherosclerosis, wherein the disease is treatable or preventable by the FIASMA and alternatively by a non-FIASMA, and wherein the non-FIASMA is not administered to the subject for the treatment or prevention of the disease. The above description in relation to the methods of treating or preventing a disease in a subject is equally applicable to these second medical use embodiments. In particular, in some embodiments, the disease is not atherosclerosis or a disease caused by atherosclerosis. In some embodiments, the method comprises not administering to the subject a non-FIASMA for the treatment or prevention of any disease or disorder.

Also described herein is a functional inhibitor of acid sphingomyelinase (FIASMA) for use in treating or preventing depression or anxiety in a subject having atherosclerosis, wherein the depression or anxiety is treatable or preventable by the FIASMA and alternatively by a non-FIASMA, and wherein the non-FIASMA is not administered to the subject for the treatment or prevention of the depression or anxiety. The above description in relation to the methods of treating or preventing a disease in a subject and the methods of treating or preventing depression or anxiety in a subject is equally applicable to these second medical use embodiments. In particular, in some embodiments, the method comprises not administering to the subject a non-FIASMA for the treatment or prevention of any disease or disorder.

### D. Further Methods

Also described herein is a method of treating or preventing a disease in a subject having atherosclerosis, wherein the disease is treatable or preventable by a functional inhibitor of acid sphingomyelinase (FIASMA) and alternatively by a non-FIASMA, wherein subject is being administered a non-FIASMA for the treatment or prevention of the disease. The method comprises ceasing administration of the non-FIASMA for the treatment or prevention of the disease, and selectively administering to the subject a FIASMA for the treatment or prevention of the disease and not administering to the subject a non-FIASMA for the treatment or prevention of the disease. The above description in relation to the methods of treating or preventing a disease in a subject is equally applicable to these embodiments.

Also described herein is a method of treating or preventing a disease in a subject having atherosclerosis, wherein the disease is treatable or preventable by a functional inhibitor of acid sphingomyelinase (FIASMA) and alternatively by a non-FIASMA. The method comprises determining that the patient is at elevated risk of MACE, and selectively administering to the subject a FIASMA for the treatment or prevention of the disease and to reduce the risk of MACE, and not administering to the subject a non-FIASMA for the treatment or prevention of the disease. In some embodiments, the term “elevated risk” means a risk above a threshold level. In some embodiments, the term “elevated risk” means that patient is at higher risk of MACE compared to a control group. In various embodiments, the control group is a cohort of healthy patients or the general population or a cohort of patients with atherosclerosis. “Elevated risk” or “higher risk” may be statistically higher risk compared to the control group. In some embodiments, the method comprises determining that the patient is at elevated risk of MACE in the next 6 months or the next two years. The above description in relation to the methods of treating or preventing a disease in a subject is equally applicable to these embodiments.

In some embodiments, determining that the patient is at elevated risk of MACE comprises determining a risk score. In some embodiments, determining that the patient is at elevated risk of MACE comprises measuring one or more biomarkers associated with MACE, and/or calculating a genetic risk score, and/or imaging the patient, and/or calculating a risk score using a machine learning risk model, and/or calculating a clinical risk score.

The one or more biomarkers associated with MACE may be any biomarker that is known to be predictive, prognostic, or associated with MACE. Various biomarkers are known in the art, for example see Wang J et al. (“Biomarker-based risk model to predict cardiovascular events in patients with acute coronary syndromes—Results from BIPass registry” The Lancet Regional Health-Western Pacific 2022; 25:100479), which is hereby incorporated by reference in its entirety. In some embodiments, the one or more biomarkers are selected from the group consisting of N-terminal pro-B-type natriuretic peptide (NT-proBNP), high-sensitivity cardiac troponin T (hs-cTnT), and growth differentiation factor 15 (GDF-15). In various embodiments, measuring the one or more biomarkers further comprises identifying that the one or more biomarkers is statistically different compared to a control. In some embodiments, measuring the one or more biomarkers further comprises calculating a biomarker risk score (e.g., BIPass risk score). Typically, the presence or amount of biomarker is determined by isolating a biological sample from a subject and detecting and/or quantifying nucleic acid encoding the biomarker and/or detecting and/or quantifying the amount of biomarker in the sample.

In various aspects, an elevated risk of MACE may be determined by calculating a genetic risk score for MACE based on sequencing of a subject's biological sample. Genetic risk score calculation is further described in, for example Ramirez J et al. (“Prediction of Coronary Artery Disease and Major Adverse Cardiovascular Events Using Clinical and Genetic Risk Scores for Cardiovascular Risk Factors”. Circulation: Genomic and Precision Medicine. 15 (5): 444-452), which is hereby incorporated by reference in its entirety.

Imaging techniques also may be employed for determining MACE risk. See for example Marcos-Garces V et al. (“Risk score for early risk prediction by cardiac magnetic resonance after acute myocardial infarction”. Int J Cardiol. 2022 Feb. 15; 349:150-154), which is hereby incorporated by reference in its entirety. In some embodiments, imaging the patient comprises imaging the patient by cardiac magnetic resonance imaging. In some embodiments, imaging the patient further comprises calculating an imaging risk score.

A risk score also may be calculated using a machine learning risk model. See for example Wang J et al. (“Risk Prediction of Major Adverse Cardiovascular Events Occurrence Within 6 Months After Coronary Revascularization: Machine Learning Study” JMIR Med Inform 2022; 10 (4): e33395), which is hereby incorporated by reference in its entirety.

An elevated risk of MACE may also be determined using a clinical risk score for MACE. The QRISK3 risk score is an exemplary method of identifying elevated risk of MACE (https://www.qrisk.org/three/). In some embodiments, calculating a clinical risk score comprises calculating a QRISK3 risk score. In various aspects, the subject demonstrates a QRISK3 score of greater than 10.

Also described herein is a method of treating or preventing a disease in a subject having atherosclerosis, wherein the disease is treatable or preventable by a functional inhibitor of acid sphingomyelinase (FIASMA) and alternatively by a non-FIASMA. The method comprises selectively administering to the subject a FIASMA for the treatment or prevention of the disease and to reduce the risk of MACE or avoid an increased risk of MACE, and not administering to the subject a non-FIASMA for the treatment or prevention of the disease. The above description in relation to the methods of treating or preventing a disease in a subject is equally applicable to these embodiments.

Also described herein is a method of reducing the risk of a MACE in a subject, wherein the subject has atherosclerosis and is being treated for a secondary disease that is not atherosclerosis. The method comprises determining that the subject is at elevated risk for MACE, and selectively administering a FIASMA for the treatment or prevention of the secondary disease and to reduce the risk of MACE, and not administering to the subject a non-FIASMA for the treatment or prevention of the secondary disease. The above description in relation to the methods of treating or preventing a disease in a subject is equally applicable to these embodiments. The above description in relation to the determination that the patient is at elevated risk of MACE is equally applicable to these embodiments.

The following examples are provided to illustrate certain particular features and/or embodiments. These examples should not be construed to limit the disclosure to the particular features or embodiments described.

### EXAMPLES

The inventors have found that patients with some degree of atherosclerosis (e.g., at least 50 years old and/or diagnosed with atherosclerosis/coronary artery disease) are at a lower risk of death and major adverse cardiac events if they receive a for the treatment of a disease instead of a non-FIASMA.

As a non-limiting example, the inventors have demonstrated that patients with presumed atherosclerosis (i.e., at least 50 years of age) receiving an anti-depression medication were at a lower risk of death and major adverse cardiac events if they received a FIASMA anti-depression medication compared to if they received a non-FIASMA anti-depression medication.

As a further non-limiting example, the inventors have demonstrated that patients with confirmed atherosclerosis (i.e., diagnosis with atherosclerosis or coronary artery disease) receiving an anti-depression medication were at a lower risk of death and major adverse cardiac events if they received a FIASMA anti-depression medication compared to if they received a non-FIASMA anti-depression medication.

These findings are important because patients with atherosclerosis (e.g., at least 50 years old and/or diagnosed with atherosclersosis/CAD) can be selectively prescribed a FIASMA over a non-FIASMA for the treatment of a disease (such as depression or anxiety) to increase their survival probability.

### Example 1

The inventors sought to determine whether there was any difference between treating patients with presumed atherosclerosis with a FIASMA versus a non-FIASMA. As a non-limiting example, the inventors selected depression/anxiety as the disease and studied the effects of the FIASMAs paroxetine and fluoxetine versus the non-FIASMAs Escitalopram and Citalopram. The ASMase inhibition data of paroxetine and fluoxetine is provided in Table 1 below. This data originates from Kornhuber J et al (Functional inhibitors of acid sphingomyelinase (FIASMAs). Handb Exp Pharmacol. 2013; (215): 169-86. doi: 10.1007/978-3-7091-1368-4_9. PMID: 23579455).

TABLE 1

| FIASMA | ATC | Log P | pKa | Residual ASMase Activity (%) |
|---|---|---|---|---|
| Paroxetine | N06AB05 | 3.9 | 10.3 | 31.7 |
| Fluoxetine | N06AB03 | 4.1 | 10.1 | 13.0 |

The analysis was conducted using the Maccabi database. The Maccabi database is an electronic medical record (EMR) database from one of the largest health maintenance organizations in Israel, representing about 26% of the total Israeli population and spanning the years 2010-2020.

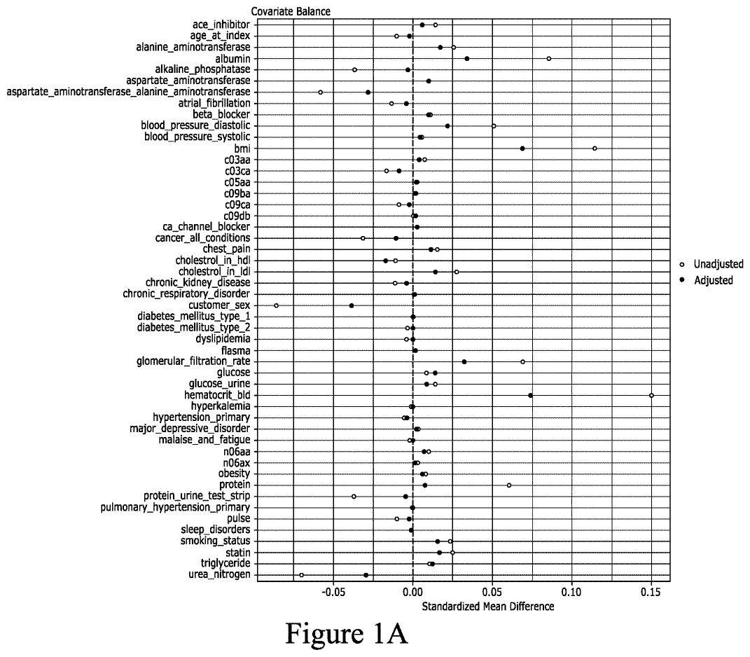

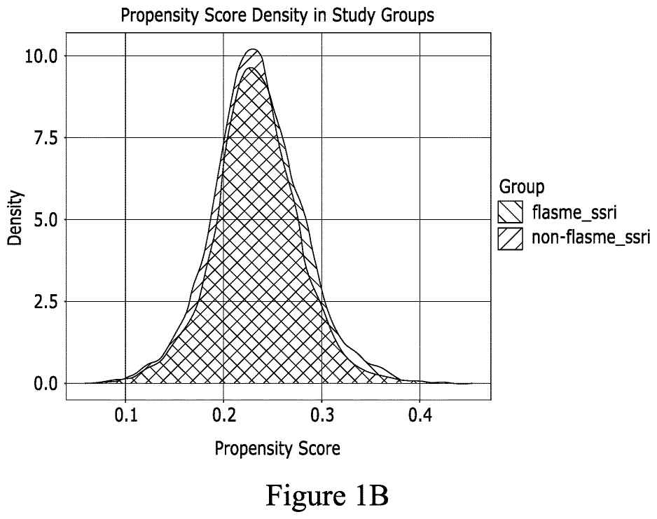

A cohort of patients (Cohort A) was defined according to Table 2 below. 13878 patients were identified from the Maccabi database who had purchased Escitalopram, Citalopram, Paroxetine, or Fluoxetine during 2012-2015, and who had not purchased any of the four SSRIs within the two years prior to the index date (i.e., the first purchase date of Escitalopram, Citalopram, Paroxetine, or Fluoxetine), and who had at least two years of activity in the database prior to the index date, and who had no myocardial infarctions two years prior to the index date, and who were aged over 50 years old. These patients were filtered based on available data for at least half of the labs and vitals, and matched on index date and propensity score, resulting in a cohort of 10363 patients with presumed atherosclerosis. A covariate balance plot and a propensity score density plot are shown in FIG. 1.

TABLE 2

| | | | Patient | Percent |
|---|---|---|---|---|
| Step | Description | Patients | Drop | Remaining |
| 1 | Patient purchased Escitalopram, Citalopram, | 77202 | 0 | 100 |
| | Paroxetine, or Fluoxetine During 2012-2015 | | | |
| | (first purchase = index date) | | | |
| 2 | Patient did not purchase any of the four SSRIs | 31999 | 58.55 | 41.45 |
| | within two years prior to index | | | |
| 3 | Patient had at least two years of activity in | 30125 | 5.86 | 39.02 |
| | Maccabi prior to index | | | |
| 4 | Patient had no MIs two years prior to index | 29927 | 0.66 | 38.76 |
| 5 | Patient aged 50 or above | 13878 | 53.63 | 17.98 |
| 6 | Patient has data for at least half of the labs & | 11514 | 17.03 | 14.91 |
| | vitals | | | |
| 6 | Matching three non-FIASMA SSRI patients to | 10363 | 10.00 | 13.42 |
| | one FIASMA patient on index date (caliper = 60 | (Cohort | | |
| | days) and propensity score (caliper = 0.1) | A) | | |

Cohort A was divided into patients who had purchased a FIASMA SSRI (Paroxetine or Fluoxetine) and patients who had purchased a non-FIASMA SSRI (Escitalopram or Citalopram). The demographics and vitals at the index date of the patients within the cohort are provided in Table 3 below.

TABLE 3

| | FIASMA | | Non-FIASMA | |
|---|---|---|---|---|
| Demographic/vital | SSRI | | SSRI | |
| Patients | 2627 | | 7736 | |
| Female Sex (n, %) | 1611 | (61.32%) | 4725 | (61.08%) |
| Age at Index (mean, SD) | 65.3 | (10.7) | 65.8 | (11.1) |
| BMI (mean, SD) | 28.52 | (5.48) | 28.15 | (5.34) |
| Systolic BP (mmHg; mean, | 129.32 | (16.82) | 129.24 | (16.74) |
| SD) | | | | |
| Diastolic BP (mmHg; mean, | 76.46 | (9.29) | 76.26 | (9.2) |
| SD) | | | | |
| Heart Rate (BPM; mean, SD) | 74.98 | (12.87) | 75.01 | (12.62) |
| Smoking Status (n, %) | | | | |
| Current smoker | 382 | (14.54%) | 1005 | (12.99%) |
| Not current smoker, unknown, | 2245 | (85.46%) | 6731 | (87.01%) |
| or missing | | | | |

The baseline comorbidities (assessed within two years prior to index) of the patients within cohort A are provided in Table 4 below.

TABLE 4

| | FIASMA | | Non-FIASMA | |
|---|---|---|---|---|
| Comorbidities (n, %) | SSRI | | SSRI | |
| Atrial Fibrillation | 104 | (3.96%) | 337 | (4.36%) |
| Cancer - All Conditions | 187 | (7.12%) | 634 | (8.2%) |
| Chest Pain | 376 | (14.31%) | 1022 | (13.21%) |
| Chronic Kidney Disease | 102 | (3.88%) | 331 | (4.28%) |
| Chronic Respiratory Disorders | 38 | (1.45%) | 107 | (1.38%) |
| Diabetes Mellitus, Type 1 | 18 | (0.69%) | 50 | (0.65%) |
| Diabetes Mellitus, Type 2 | 309 | (11.76%) | 907 | (11.72%) |
| Dyslipidemia | 83 | (3.16%) | 250 | (3.23%) |
| Hypertension, Primary | 336 | (12.79%) | 1019 | (13.17%) |
| Major Depressive Disorder | 22 | (0.84%) | 46 | (0.59%) |
| Malaise and Fatigue | 9 | (0.34%) | 33 | (0.43%) |
| Obesity | 124 | (4.72%) | 317 | (4.10%) |
| Pulmonary Hypertension, Primary | 2 | (0.08%) | 6 | (0.08%) |
| Sleep Disorders | 79 | (3.01%) | 241 | (3.12%) |

The baseline labs (identified as the closest value prior to index within one year) of the of the patients within cohort A are provided in Table 5 below.

TABLE 5

| Labs (mean, SD) | FIASMA SSRI | | Non-FIASMA SSRI | |
|---|---|---|---|---|
| Cholesterol in HDL | 51.97 | (13.78) | 52.21 | (13.95) |
| Cholesterol in LDL | 115.53 | (35.08) | 115.04 | (35.19) |
| Hematocrit Blood | 41.33 | (3.95) | 41.03 | (4.05) |
| Triglyceride | 133.08 | (62.53) | 132.34 | (62.03) |
| Urea nitrogen | 35.26 | (12.51) | 35.63 | (12.72) |
| Albumin | 4.24 | (0.34) | 4.22 | (0.36) |
| Protein | 7.14 | (0.47) | 7.14 | (0.49) |
| Alkaline phosphatase | 84.92 | (31.39) | 85.03 | (32.32) |
| Glomerular filtration rate | 88.71 | (22.83) | 87.96 | (23.23) |
| Glucose | 107.24 | (24.64) | 106.89 | (24.44) |
| Glucose Urine | 6.18 | (40.09) | 5.84 | (38.71) |
| Protein Urine Test strip | 9.67 | (21.14) | 9.77 | (22.05) |
| Alanine aminotransferase | 21.43 | (11.98) | 21.22 | (11.91) |
| Aspartate aminotransferase | 23.15 | (8.86) | 23.06 | (8.7) |
| Aspartate aminotransferase/ | 1.21 | (0.4) | 1.22 | (0.42) |
| Alanine aminotransferase | | | | |

The baseline medications of the of the patients within cohort A are provided in Table 6 below.

TABLE 6

| | FIASMA | | Non-FIASMA | |
|---|---|---|---|---|
| Medication usage (n, %) | SSRI | | SSRI | |
| Statins | 1442 | (54.89%) | 4119 | (53.24%) |
| ACE inhibitors | 678 | (25.81%) | 1951 | (25.22%) |
| Beta blockers | 878 | (33.42%) | 2509 | (32.43%) |
| Calcium Channel Blockers | 639 | (24.32%) | 1860 | (24.04%) |
| C03AA: Thiazides, plain | 193 | (7.35%) | 538 | (6.95%) |
| C03CA: Sulfonamides, plain | 177 | (6.74%) | 588 | (7.6%) |
| C05AA: Corticosteroids | 28 | (1.07%) | 67 | (0.87%) |
| C09BA: ACE inhibitors and | 186 | (7.08%) | 537 | (6.94%) |
| diuretics | | | | |
| C09CA: Angiotensin II receptor | 371 | (14.12%) | 1111 | (14.36%) |
| blockers, plain | | | | |
| C09DB: Angiotensin II receptor | 123 | (4.68%) | 349 | (4.51%) |
| blockers and Ca channel blockers | | | | |
| N06AA: Non-selective | 143 | (5.44%) | 367 | (4.74%) |
| monoamine reuptake inhibitors | | | | |
| N06AX: Other antidepressants | 324 | (12.33%) | 944 | (12.2%) |
| Non-SSRI FIASMA | 738 | (28.09%) | 2159 | (27.91%) |

Univariate Cox Proportional Hazards regression analysis was used to investigate the association between the survival time of the subjects and whether they had been receiving a FIASMA or a non-FIASMA SSRI. Cox Proportional Hazards models are semi-parametric and do not make assumptions about the distribution of the baseline hazard function; a key assumption that is made is that hazard function between strata remains proportional over time.

$$h ( t ) = {h}_{0} ( t ) exp ( {B}_{1} {x}_{1} + … + {B}_{p} {x}_{p} ) \\ {h}_{0} is the baseline hazard rate$$

The following survival assumptions were made:

Survival Assumption 1 (with No Treatment Length Data)

### Non-Death Outcomes

### Coding Days of Follow-Up:

Patient had an event: time from index to event
Patient had NO event:
   Patient died during follow-up: time from index to death
   Patient did NOT die during follow-up: time from index to max activity date (where max activity date=the latest date of a coded diagnosis/procedure or death date (pre-2021))

Three patients were removed who had no event and a max activity date before index.
Events that occurred within 90 days of index were ignored.

Coding event indicator: 1 if event happened during follow-up, 0 otherwise.

### All-Cause Mortality

### Coding Days of Follow-Up:

Patient had an event: time from index to death.
Patient had NO event: time from index to max activity date (where max activity date=the latest date of a coded diagnosis/procedure (pre-2021))

Three patients were removed who had no event and a max activity date before index.
Events that occurred within 90 days of index were ignored.

Coding event indicator: 1 if death happened during follow-up, 0 otherwise.

Survival Assumption 2 (with Treatment Length Data)

### Non-Death Outcomes

### Coding Days of Follow-Up:

Patient had an event: time from index to event
Patient had NO event:
   Patient died during follow-up: time from index to the earlier of 1) end of treatment, 2) death date
   Patient did NOT die during follow-up: time from index to end of treatment

Three patients were removed who had no event and a max activity date before index. Events that occurred within 90 days of index were ignored.

Coding event indicator: 1 if event happened during SSRI usage or within a year of stoppage, 0 otherwise.

### All-Cause Mortality

### Coding Days of Follow-Up:

Patient had an event: time from index to death.
Patient had NO event: time from index to end of treatment
   Three patients were removed who had no event and a max activity date before index.
Events that occurred within 90 days of index were ignored.

Coding event indicator: 1 if death happened during SSRI usage or within a year of stoppage, 0 otherwise.

The results of the Univariate Cox Proportional Hazards regression analysis are provided in Table 7 below.

TABLE 7

| | Survival Assumption 1 | | | Survival Assumption 2 | | |
|---|---|---|---|---|---|---|
| Outcome | HR | 95% CI | p-value | HR | 95% CI | p-value |
| All-cause Mortality | 0.91 | (0.83, 1.01) | 0.09 | 0.71 | (0.61, 0.83) | 1.01E−05 |
| MACE (combination of all- | 0.94 | (0.87, 1.02) | 0.14 | 0.79 | (0.70, 0.89) | 6.33E−05 |
| cause mortality, acute MI, | | | | | | |
| stroke and revascularization) | | | | | | |

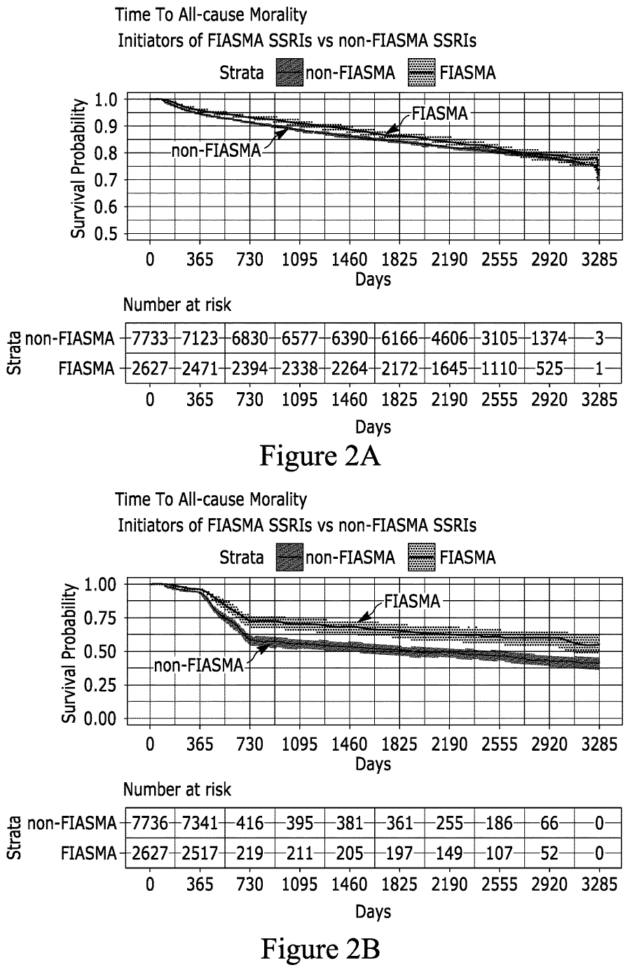

A factor that reached statistical significance under survival assumption 2 was all-cause mortality (HR=0.71; 95% CI 0.61-0.83; p-value 1.01E-05). This is also evident from the Kaplan Meier survival curve shown in FIG. 2B. Among patients with presumed atherosclerosis (i.e., over the age of 50), FIASMA SSRI use was associated with a lower risk of all-cause mortality relative to non-FIASMA SSRI use, and this effect was sustained over the study period.

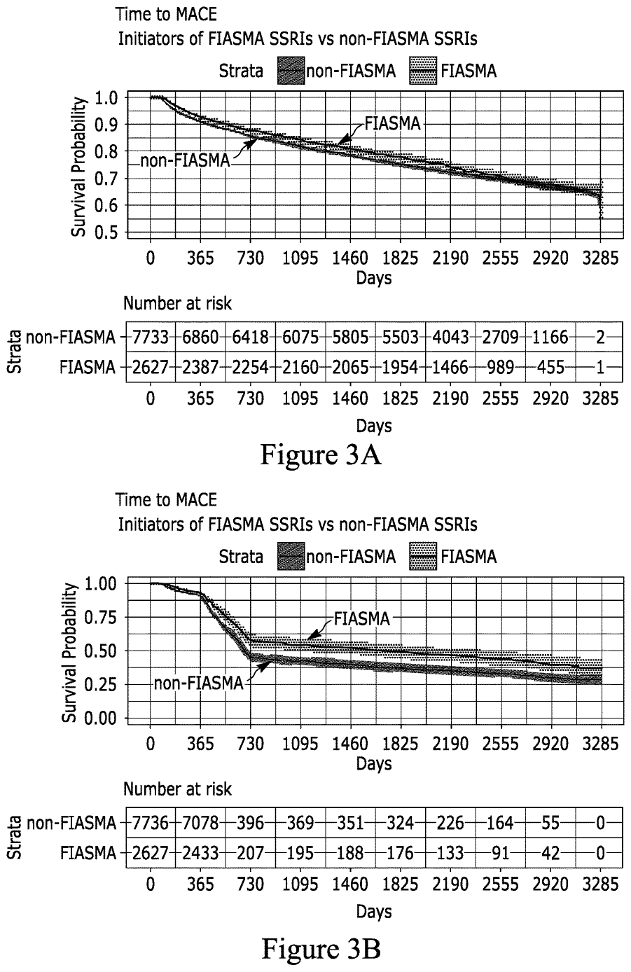

A further factor that reached statistical significance under survival assumption 2 was MACE (HR=0.79; 95% CI 0.70-0.89; p-value 6.33E-05). This is evident from the Kaplan Meier survival curve shown in FIG. 3B. Among patients with presumed atherosclerosis (i.e., over the age of 50), FIASMA SSRI use was associated with a lower risk of a major adverse cardiac event relative to non-FIASMA SSRI use, and this effect was sustained over the study period.

### Example 2

The inventors went on to determine whether there was any difference between treating patients with confirmed atherosclerosis/coronary artery disease with a FIASMA SSRI versus a non-FIASMA SSRI.

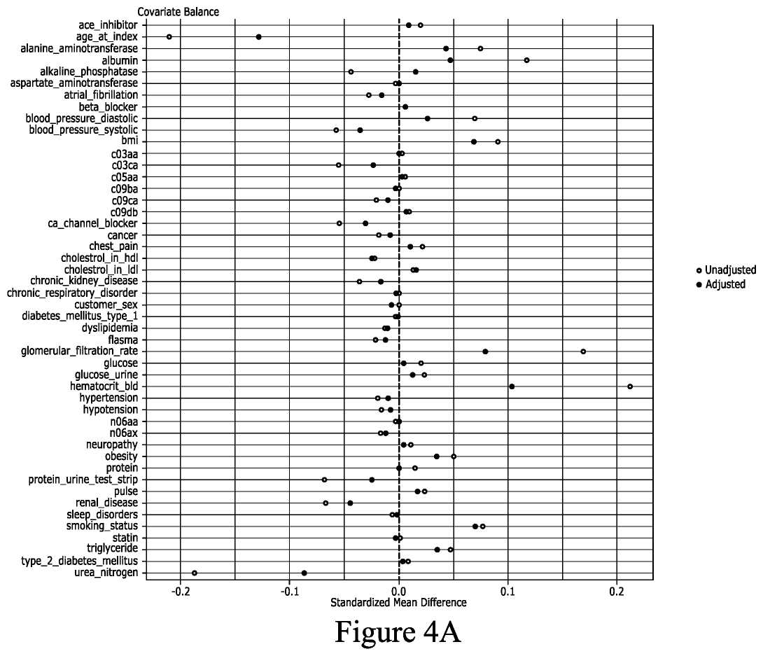

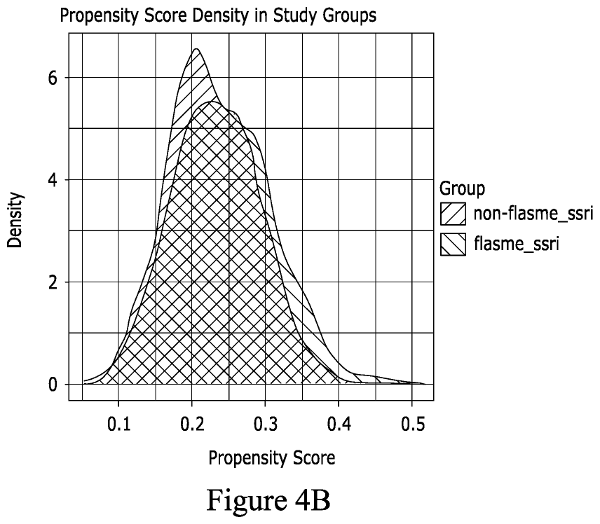

The analysis was conducted using the Maccabi database. A cohort of patients (cohort B) was defined according to Table 8 below. 3050 patients were identified from the Maccabi database who had purchased Escitalopram, Citalopram, Paroxetine, or Fluoxetine during 2012-2015, and who had not purchased any of the four SSRIs within one year prior to the index date, and who had at least two years of activity in the database prior to the index date, and who had been diagnosed with coronary artery disease or atherosclerosis within ten years of the index date. These patients were filtered based on available data for at least half of the labs and vitals, and matched on index date and propensity score, resulting in a cohort of 2486 patients with confirmed atherosclerosis/coronary artery disease. A covariate balance plot and a propensity score density plot are shown in FIG. 4.

TABLE 8

| | | | Percent | Percent |
|---|---|---|---|---|
| Step | Description | Patients | Drop | Remaining |
| 1 | Patient purchased Escitalopram, Citalopram, | 77202 | 0 | 100 |
| | Paroxetine, or Fluoxetine During Jan. 1, 2012- | | | |
| | Dec. 31, 2015 | | | |
| 2 | Patient did not purchase any of the four SSRIs | 33479 | 56.63 | 43.37 |
| | within one year prior to index | | | |
| 3 | Patient had at least two years of activity in | 31565 | 5.72 | 40.89 |
| | Maccabi prior to index | | | |
| 4 | Patient diagnosed with CAD or atherosclerosis | 3050 | 90.34 | 3.95 |
| | within ten years of index | | | |
| 5 | Patient has data for at least half of the labs & | 2822 | 7.48 | 3.66 |
| | vitals | | | |
| 6 | Matching on index date (caliper = 60) and | 2486 | 11.91 | 3.22 |
| | propensity score (caliper = 0.1) | (Cohort | | |
| | | B) | | |

74.5% of patients in cohort B (with confirmed atherosclerosis/coronary artery disease) were present in cohort A of Example 1 (with presumed atherosclerosis).

Cohort B was divided into patients who had purchased a FIASMA SSRI (Paroxetine or Fluoxetine) and patients who had purchased a non-FIASMA SSRI (Escitalopram or Citalopram). The demographics and vitals at the index date of the patients within cohort B are provided in Table 9 below.

TABLE 9

| | FIASMA | | Non-FIASMA | |
|---|---|---|---|---|
| | SSRI | | SSRI | |
| Patients | 632 | | 1854 | |
| Female Sex (n, %) | 238 | (37.7%) | 686 | (37.0%) |
| Age at Index (mean, SD) | 67.9 | (12.7) | 69.6 | (12.6) |
| BMI (mean, SD) | 28.89 | (5.12) | 28.54 | (5.07) |
| Systolic BP (mmHG; mean, | 130.21 | (17.02) | 130.82 | (17.52) |
| SD) | | | | |
| Diastolic BP (mmHg; mean, | 75.51 | (9.65) | 75.24 | (9.73) |
| SD) | | | | |
| Heart Rate (BPM; mean, SD) | 73.09 | (12.57) | 72.87 | (12.62) |
| Smoking Status (n, %) | | | | |
| Current smoker | 126 | (19.94%) | 240 | (12.94%) |
| Not current smoker, unknown, | 506 | (80.06%) | 1614 | (87.06%) |
| or missing | | | | |

The baseline comorbidities (assessed within two years prior to index) of the patients within cohort B are provided in Table 10 below.

TABLE 10

| Comorbidities (n, %) | FIASMA SSRI | | Non-FIASMA SSRI | |
|---|---|---|---|---|
| Atrial Fibrillation | 84 | (13.29%) | 275 | (14.83%) |
| Cancer | 28 | (4.43%) | 97 | (5.23%) |
| Chest Pain | 224 | (35.44%) | 638 | (34.41%) |
| Chronic Kidney Disease | 71 | (11.23%) | 238 | (12.84%) |
| Chronic Respiratory Disorders | 21 | (3.32%) | 66 | (3.56%) |
| Diabetes Mellitus, Type 1 | <20 | (1.74%) | 34 | (1.83%) |
| Diabetes Mellitus, Type 2 | 262 | (41.46%) | 761 | (41.05%) |
| Dyslipidemia | 526 | (83.23%) | 1564 | (84.36%) |
| Hypertension | 517 | (81.8%) | 1534 | (82.74%) |
| Hypotension | 34 | (5.38%) | 113 | (6.09%) |
| Neuropathy | 40 | (6.33%) | 108 | (5.83%) |
| Obesity | 189 | (29.91%) | 490 | (26.43%) |
| Renal Disease | 452 | (71.52%) | 1407 | (75.89%) |
| Sleep Disorders | 27 | (4.27%) | 81 | (4.37%) |

The baseline labs (identified as the closest value prior to index within one year) of the of the patients within cohort B are provided in Table 11 below.

TABLE 11

| Labs (mean, SD) | FIASMA SSRI | | Non-FIASMA SSRI | |
|---|---|---|---|---|
| Cholesterol in HDL | 46.42 | (12.41) | 46.73 | (12.5) |
| Cholesterol in LDL | 97.04 | (34.25) | 96.47 | (35.48) |
| Hematocrit Blood | 41.32 | (4.62) | 40.84 | (4.59) |
| Triglyceride | 139.56 | (68.79) | 137.17 | (67.55) |
| Urea nitrogen | 39.69 | (17.2) | 41.22 | (18.11) |
| Albumin | 4.15 | (0.37) | 4.13 | (0.41) |
| Protein | 7.05 | (0.53) | 7.05 | (0.54) |
| Alkaline phosphatase | 87.72 | (34.63) | 87.17 | (35.24) |
| Glomerular filtration rate | 84.05 | (26.35) | 81.95 | (26.07) |
| Glucose | 113.7 | (30.05) | 113.55 | (31.39) |
| Glucose Urine | 13.6 | (62.93) | 12.79 | (64.77) |
| Protein Urine Test strip | 14.17 | (31.97) | 14.98 | (33.91) |
| Alanine aminotransferase | 22.83 | (15.43) | 22.19 | (13.97) |
| Aspartate aminotransferase | 23.68 | (9.89) | 23.67 | (9.08) |

The baseline medications (assessed within one year prior to index) of the patients within cohort B are provided in Table 12 below.

TABLE 12

| | FIASMA | | Non-FIASMA | |
|---|---|---|---|---|
| Medication usage (n, %) | SSRI | | SSRI | |
| Statins | 505 | (79.91%) | 1487 | (80.2%) |
| ACE inhibitors | 250 | (39.56%) | 716 | (38.62%) |
| Beta blockers | 406 | (64.24%) | 1179 | (63.59%) |
| Calcium Channel Blockers | 214 | (33.86%) | 685 | (36.95%) |
| C03AA: Thiazides, plain | 52 | (8.23%) | 151 | (8.14%) |
| C03CA: Sulfonamides, plain | 101 | (15.98%) | 340 | (18.34%) |
| C05AA: Corticosteroids | 8 | (1.27%) | 16 | (0.86%) |
| C09BA: ACE inhibitors and | 51 | (8.07%) | 155 | (8.36%) |
| diuretics | | | | |
| C09CA: Angiotensin II receptor | 144 | (22.78%) | 440 | (23.73%) |
| blockers, plain | | | | |
| C09DB: Angiotensin II receptor | 47 | (7.44%) | 127 | (6.85%) |
| blockers and calcium channel | | | | |
| blockers | | | | |
| N06AA: Non-selective | 24 | (3.8%) | 67 | (3.61%) |
| monoamine reuptake inhibitors | | | | |
| N06AX: Other antidepressants | 68 | (10.76%) | 221 | (11.92%) |
| Non-SSRI FIASMA | 181 | (28.64%) | 553 | (29.83%) |

Univariate Cox Proportional Hazards regression analysis was used to investigate the association between the survival time of the subjects and whether they had been receiving a FIASMA or a non-FIASMA SSRI. Survival assumptions 1 and 2 of Example 1 were used. The results of this analysis are provided in Table 13 below.

TABLE 13

| | Survival Assumption 1 | | | Survival Assumption 2 | | |
|---|---|---|---|---|---|---|
| Outcome | HR | 95% CI | p-value | HR | 95% CI | p-value |
| All-cause Mortality | 0.85 | (0.72, 1.01) | 0.06 | 0.60 | (0.47, 0.76) | 4.32E−05 |
| MACE (combination of all- | 0.88 | (0.78, 0.99) | 0.04 | 0.73 | (0.62, 0.85) | 4.03E−05 |
| cause mortality, acute MI, | | | | | | |
| stroke and revascularization) | | | | | | |

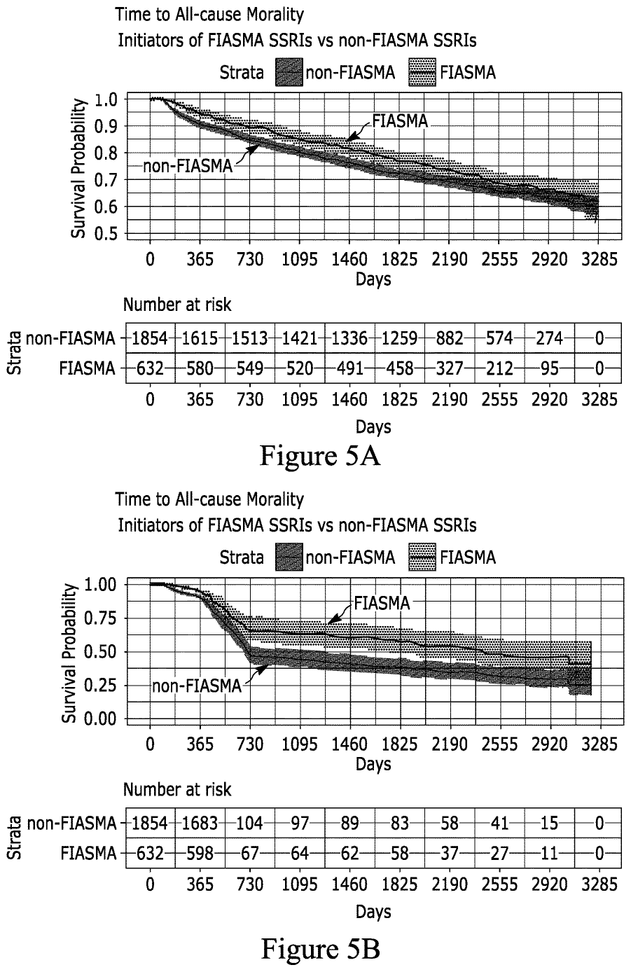

A factor that reached statistical significance under survival assumption 2 was all-cause mortality (hazard ratio (HR), 0.60; 95% confidence interval (CI) 0.47-0.76; p=4.32E-05). This is also evident from the Kaplan Meier survival curve shown in FIG. 5B. Among patients with confirmed atherosclerosis/CAD, FIASMA SSRI use was associated with a lower risk of all-cause mortality relative to non-FIASMA SSRI use, and this effect was sustained over the study period.

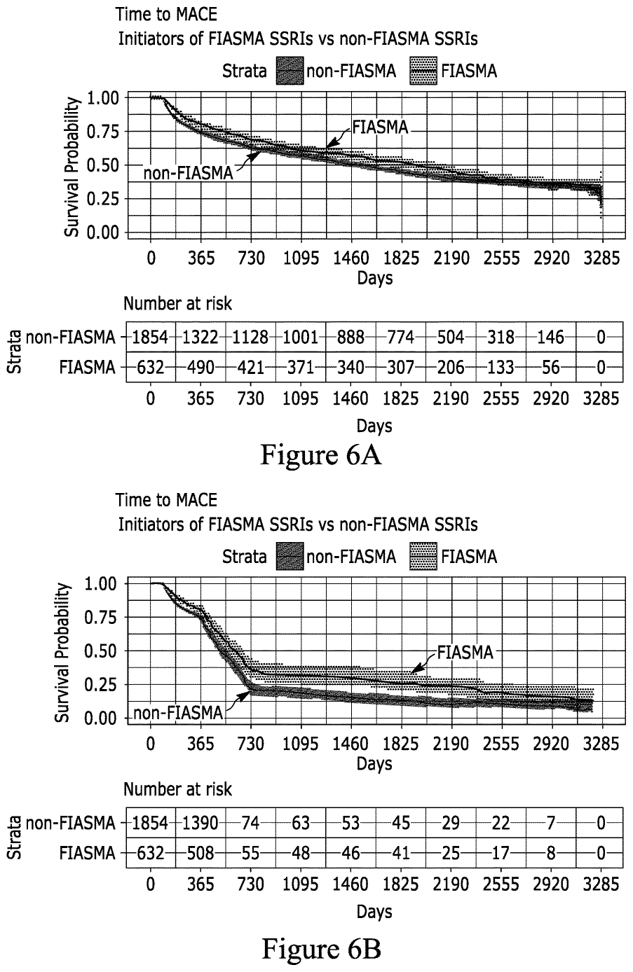

A factor that reached statistical significance under survival assumptions 1 and 2 was MACE. This is also evident from the Kaplan Meier survival curves shown in FIGS. 6A and 6B.

The results from the confirmed atherosclerosis/CAD cohort B corroborates the results from the presumed atherosclerosis cohort A of Example 1; FIASMAs have a significant protective effect on all-cause mortality and major adverse cardiac events relative to non-FIASMAs.

In view of the many possible embodiments to which the principles of the disclosed invention may be applied, it should be recognised that the illustrated embodiments are only preferred examples of the invention and should not be taken as limiting the scope of the invention. Rather, the scope of the invention is defined by the following claims. We therefore claim as our invention all that comes within the scope and spirit of these claims.

### REFERENCES

1. Kornhuber J, Tripal P, Gulbins E, Muehlbacher M. Functional inhibitors of acid sphingomyelinase (FIASMAS). Handb Exp Pharmacol. 2013; (215): 169-186. doi: 10.1007/978-3-7091-1368-4_9
2. Kornhuber J, Muehlbacher M, Trapp S, Pechmann S, Friedl A, Mühle C, Terfloth L, Groemer T W, Spitzer G M, Liedl K R, Gulbins E, Tripal P. Identification of Novel Functional Inhibitors of Acid Sphingomyelinase. PLOS ONE 2011; 6 (8): e23852. https://doi.org/10.1371/journal.pone.0023852

1. A method of treating or preventing a disease in a subject having atherosclerosis, wherein the disease is treatable or preventable by a functional inhibitor of acid sphingomyelinase (FIASMA) and alternatively by a non-FIASMA, wherein the method comprises:
selectively administering to the subject a FIASMA for the treatment or prevention of the disease and not administering to the subject a non-FIASMA for the treatment or prevention of the disease.

2. A method according to claim 1, wherein the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

3. A method according to claim 2, wherein the disease is depression or anxiety.

4. A method according to claim 3, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

5. A method according to any one of claims 1-4, wherein the subject is at least 50 years old and/or the subject has family history of myocardial infarction and/or the subject has previously had a myocardial infarction.

6. A method according to any one of claims 1-5, wherein the subject has coronary artery disease.

7. A method according to any preceding claim, wherein the method comprises not administering to the subject a non-FIASMA for the treatment or prevention of any disease or disorder.

8. A method according to any preceding claim, wherein the disease is not atherosclerosis or a disease caused by atherosclerosis.

9. A method according to any preceding claim, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

10. A method according to claim 9, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

11. A method according to any preceding claim, wherein the FIASMA is a selective serotonin reuptake inhibitor.

12. A method according to any preceding claim, wherein the FIASMA is Paroxetine or Fluoxetine.

13. A method according to any preceding claim, wherein the subject is a non-human animal.

14. A method according to any one of claims 1-12, wherein the subject is human.

15. A method of treating or preventing depression or anxiety in a subject having atherosclerosis, wherein the depression or anxiety is treatable or preventable by a functional inhibitor of acid sphingomyelinase (FIASMA) and alternatively by a non-FIASMA, wherein the method comprises:
selectively administering to the subject a FIASMA for the treatment or prevention of the depression or anxiety and not administering to the subject a non-FIASMA for the treatment or prevention of the depression or anxiety.

16. A method according to claim 15, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

17. A method according to claim 15 or claim 16, wherein the subject is at least 50 years old and/or the subject has family history of myocardial infarction and/or the subject has previously had a myocardial infarction.

18. A method according to any one of claims 15-17, wherein the subject has coronary artery disease.

19. A method according to any one of claims 15-18, wherein the method comprises not administering to the subject a non-FIASMA for the treatment or prevention of any disease or disorder.

20. A method according to any one of claims 15-19, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

21. A method according to any one of claims 15-20, wherein the FIASMA is a selective serotonin reuptake inhibitor.

22. A method according to any one of claims 15-21, wherein the FIASMA is Paroxetine or Fluoxetine.

23. A method according to any one of claims 15-22, wherein the subject is a non-human animal.

24. A method according to any one of claims 15-22, wherein the subject is human.

25. A functional inhibitor of acid sphingomyelinase (FIASMA) for use in treating or preventing a disease in a subject having atherosclerosis,
wherein the disease is treatable or preventable by the FIASMA and alternatively by a non-FIASMA, and
wherein the non-FIASMA is not administered to the subject for the treatment or prevention of the disease.

26. The FIASMA for use according to claim 25, wherein the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

27. The FIASMA for use according to claim 25 or claim 26, wherein the disease is depression or anxiety.

28. The FIASMA for use according to any one of claims 25-27, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

29. The FIASMA for use according to any one of claims 25-28, wherein the subject is at least 50 years old and/or the subject has family history of myocardial infarction and/or the subject has previously had a myocardial infarction.

30. The FIASMA for use according to any one of claims 25-29, wherein the subject has coronary artery disease.

31. The FIASMA for use according to any one of claims 25-30, wherein the subject is not administered a non-FIASMA for the treatment or prevention of any disease or disorder.

32. The FIASMA for use according to any one of claims 25-31, wherein the disease is not atherosclerosis or a disease caused by atherosclerosis.

33. The FIASMA for use according to any one of claims 25-32, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

34. The FIASMA for use according to any one of claims 25-33, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

35. The FIASMA for use according to any one of claims 25-34, wherein the FIASMA is a selective serotonin reuptake inhibitor.

36. The FIASMA for use according to any one of claims 25-35, wherein the FIASMA is Paroxetine or Fluoxetine.

37. The FIASMA for use according to any one of claims 25-36, wherein the subject is a non-human animal.

38. The FIASMA for use according to any one of claims 25-36, wherein the subject is human.

39. A functional inhibitor of acid sphingomyelinase (FIASMA) for use in treating or preventing depression or anxiety in a subject having atherosclerosis,
wherein the depression or anxiety is treatable or preventable by the FIASMA and alternatively by a non-FIASMA, and
wherein the non-FIASMA is not administered to the subject for the treatment or prevention of the depression or anxiety.

40. The FIASMA for use according to claim 39, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

41. The FIASMA for use according to claim 39 or 40, wherein the subject is at least 50 years old and/or the subject has family history of myocardial infarction and/or the subject has previously had a myocardial infarction.

42. The FIASMA for use according to any one of claims 39-41, wherein the subject has coronary artery disease.

43. The FIASMA for use according to any one of claims 39-42, wherein the subject is not administered a non-FIASMA for the treatment or prevention of any disease or disorder.

44. The FIASMA for use according to any one of claims 39-43, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

45. The FIASMA for use according to any one of claims 39-44, wherein the FIASMA is a selective serotonin reuptake inhibitor.

46. The FIASMA for use according to any one of claims 39-45, wherein the FIASMA is Paroxetine or Fluoxetine.

47. The FIASMA for use according to any one of claims 39-46, wherein the subject is a non-human animal.

48. The FIASMA for use according to any one of claims 39-46, wherein the subject is human.

49. A method of treating or preventing a disease in a subject having atherosclerosis, wherein the disease is treatable or preventable by a functional inhibitor of acid sphingomyelinase (FIASMA) and alternatively by a non-FIASMA, wherein subject is being administered a non-FIASMA for the treatment or prevention of the disease, wherein the method comprises:
ceasing administration of the non-FIASMA for the treatment or prevention of the disease, and
selectively administering to the subject a FIASMA for the treatment or prevention of the disease and not administering to the subject a non-FIASMA for the treatment or prevention of the disease.

50. A method according to claim 49, wherein the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

51. A method according to claim 49 or claim 50, wherein the disease is depression or anxiety.

52. A method according to any one of claims 49-51, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

53. A method according to any one of claims 49-52, wherein the subject is at least 50 years old and/or the subject has family history of myocardial infarction and/or the subject has previously had a myocardial infarction.

54. A method according to any one of claims 49-53, wherein the subject has coronary artery disease.

55. A method according to any one of claims 49-54, wherein the method comprises not administering to the subject a non-FIASMA for the treatment or prevention of any disease or disorder.

56. A method according to any one of claims 49-55, wherein the disease is not atherosclerosis or a disease caused by atherosclerosis.

57. A method according to any one of claims 49-56, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

58. A method according to any one of claims 49-57, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

59. A method according to any one of claims 49-58, wherein the FIASMA is a selective serotonin reuptake inhibitor.

60. A method according to any one of claims 49-59, wherein the FIASMA is Paroxetine or Fluoxetine.

61. A method according to any one of claims 49-60, wherein the subject is a non-human animal.

62. A method according to any one of claims 49-60, wherein the subject is human.

63. A method of treating or preventing a disease in a subject having atherosclerosis, wherein the disease is treatable or preventable by a functional inhibitor of acid sphingomyelinase (FIASMA) and alternatively by a non-FIASMA, wherein the method comprises:
determining that the patient is at elevated risk of MACE, and
selectively administering to the subject a FIASMA for the treatment or prevention of the disease and to reduce the risk of MACE, and not administering to the subject a non-FIASMA for the treatment or prevention of the disease.

64. A method according to claim 63, wherein the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

65. A method according to claim 63 or claim 64, wherein the disease is depression or anxiety.

66. A method according to any one of claims 63-65, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

67. A method according to any one of claims 63-66, wherein the subject is at least 50 years old and/or the subject has family history of myocardial infarction and/or the subject has previously had a myocardial infarction.

68. A method according to any one of claims 63-67, wherein the subject has coronary artery disease.

69. A method according to any one of claims 63-68, wherein the method comprises not administering to the subject a non-FIASMA for the treatment or prevention of any disease or disorder.

70. A method according to any one of claims 63-69, wherein the disease is not atherosclerosis or a disease caused by atherosclerosis.

71. A method according to any one of claims 63-70, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

72. A method according to any one of claims 63-71, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

73. A method according to any one of claims 63-72, wherein the FIASMA is a selective serotonin reuptake inhibitor.

74. A method according to any one of claims 63-73, wherein the FIASMA is Paroxetine or Fluoxetine.

75. A method according to any one of claims 63-74, wherein the subject is a non-human animal.

76. A method according to any one of claims 63-74, wherein the subject is human.

77. A method of treating or preventing a disease in a subject having atherosclerosis, wherein the disease is treatable or preventable by a functional inhibitor of acid sphingomyelinase (FIASMA) and alternatively by a non-FIASMA, wherein the method comprises:
selectively administering to the subject a FIASMA for the treatment or prevention of the disease and to reduce the risk of MACE or avoid an increased risk of MACE, and not administering to the subject a non-FIASMA for the treatment or prevention of the disease.

78. A method according to claim 77, wherein the disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

79. A method according to claim 77 or claim 78, wherein the disease is depression or anxiety.

80. A method according to any one of claims 77-79, wherein the disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

81. A method according to any one of claims 77-80, wherein the subject is at least 50 years old and/or the subject has family history of myocardial infarction and/or the subject has previously had a myocardial infarction.

82. A method according to any one of claims 77-81, wherein the subject has coronary artery disease.

83. A method according to any one of claims 77-82, wherein the method comprises not administering to the subject a non-FIASMA for the treatment or prevention of any disease or disorder.

84. A method according to any one of claims 77-83, wherein the disease is not atherosclerosis or a disease caused by atherosclerosis.

85. A method according to any one of claims 77-84, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

86. A method according to any one of claims 77-85, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

87. A method according to any one of claims 77-86, wherein the FIASMA is a selective serotonin reuptake inhibitor.

88. A method according to any one of claims 77-87, wherein the FIASMA is Paroxetine or Fluoxetine.

89. A method according to any one of claims 77-88, wherein the subject is a non-human animal.

90. A method according to any one of claims 77-88, wherein the subject is human.

91. A method of reducing the risk of a MACE in a subject,
wherein the subject has atherosclerosis and is being treated for a secondary disease that is not atherosclerosis, wherein the method comprises:
determining that the subject is at elevated risk for MACE, and
selectively administering a FIASMA for the treatment or prevention of the secondary disease and to reduce the risk of MACE, and not administering to the subject a non-FIASMA for the treatment or prevention of the secondary disease.

92. A method according to claim 91, wherein the secondary disease is selected from the group consisting of: depression, anxiety, allergy, arrhythmia, pain, angina, hypertension, psychosis, schizophrenia, mania, muscle spasms, pruritus, bulimia nervosa, premenstrual dysphoric disorder, alcohol abuse, cystic fibrosis, septic shock, microbial infection and irritable bowel syndrome.

93. A method according to claim 91 or claim 92, wherein the secondary disease is depression or anxiety.

94. A method according to any one of claims 91-93, wherein the secondary disease is depression, and wherein the depression is selected from the group consisting of: clinical depression, depressive episode, recurrent depressive disorder, reactive depression, dysthymia, cyclothymia, manic depression, psychotic depression, prenatal depression, postnatal depression and seasonal affective disorder; or wherein the disease is anxiety, and wherein the anxiety is selected from the group consisting of: generalised anxiety disorder, obsessive-compulsive disorder, panic disorder, post-traumatic stress disorder and social phobia.

95. A method according to any one of claims 91-94, wherein the subject is at least 50 years old and/or the subject has family history of myocardial infarction and/or the subject has previously had a myocardial infarction.

96. A method according to any one of claims 91-95, wherein the subject has coronary artery disease.

97. A method according to any one of claims 91-96, wherein the method comprises not administering to the subject a non-FIASMA for the treatment or prevention of any disease or disorder.

98. A method according to any one of claims 91-97, wherein the secondary disease is not atherosclerosis or a disease caused by atherosclerosis.

99. A method according to any one of claims 91-98, wherein the FIASMA is selected from the group consisting of: Alverine, Amiodarone, Amitriptyline, Amlodipine, Aprindine, Astemizole, AY-9944, Benztropine, Bepridil, Biperiden, Camylofine, Carvedilol, Cepharanthine, Chlorpromazine, Chlorprothixene, Cinnarizine, Clemastin, Clofazimine, Clomiphene, Clomipramine, Cloperastine, Connesine, Cyclobenzaprine, Cyproheptadine, Desipramine, Desloratadine, Dicyclomine, Dilazep, Dimebon, Doxepin, Drofenine, Emetine, Ethopropazine, Fendiline, Flunarizine, Fluoxetine, Flupenthixol, Fluphenazine, Fluvoxamine, Hydroxyzine, Imipramine, Lofepramine, Loperamid, Loratadine, Maprotiline, Mebeverine, Mebhydroline, Mibefradil, Norfluoxetine, Nortriptyline, Paroxetine, Penfluridol, Perhexiline, Perphenazine, Pimethixene, Pimozide, Promazin, Promethazin, Protriptyline, Quinacrine, Sertindole, Sertraline, Solasodine, Suloctidil, Tamoxifen, Terfenadine, Thioridazin, Tomatidine, Trifluoperazine, Triflupromazine, Trimipramine, Zolantidine, and combinations thereof.

100. A method according to any one of claims 91-99, wherein the FIASMA is selected from the group consisting of: Amitriptyline, Clomipramine, Desipramine, Doxepin, Fluoxetine, Flupenthixol, Fluvoxamine, Imipramine, Lofepramine, Maprotiline, Norfluoxetine, Nortriptyline, Paroxetine, Protriptyline, Sertraline, and Trimipramine.

101. A method according to any one of claims 91-100, wherein the FIASMA is a selective serotonin reuptake inhibitor.

102. A method according to any one of claims 91-101, wherein the FIASMA is Paroxetine or Fluoxetine.

103. A method according to any one of claims 91-102, wherein the subject is a non-human animal.

104. A method according to any one of claims 91-102, wherein the subject is human.
